SEQUENCE LISTING (3 sequences)

SEQ ID NO: 1 - DNA, 168 bp, Parascedosporium putredinis NO1
cctatgttctgttgtctcggcggggcgccaggcctttggccaaacccccgccggcagcataaaactcttatattttatagcggattatatttctgaattaaacacaaaacaaatcaaaactttcaacaacggatctcttggttctggcatcgatgaagaacgcagcga

SEQ ID NO: 2 - DNA, 19 bp, artificial sequence
  primer
tccgtaggtgaacctgcgg

SEQ ID NO: 3 - DNA, 17 bp, artificial sequence
  primer
cgctgcgttcttcatcg

(19) United States
(12) Patent Application Publication
(10) Pub. No.: US 2024/0358043 A1
(43) Pub. Date: Oct. 31, 2024

(54) INSECT BIOMASS PREPARATION

(71) Applicant: The University of York, York, Yorkshire (GB)

(72) Inventors: Neil Bruce, York (GB); Alexander Setchfield, York (GB); Daniel Leadbeater, York (GB)

(73) Assignee: The University of York, York, Yorkshire (GB)

(21) Appl. No.: 18/287,731

(22) PCT Filed: Apr. 21, 2022
(86) PCT No.: PCT/EP2022/060576

§ 371 Date: Oct. 20, 2023

(30) Foreign Application Priority Data
Apr. 23, 2021 (GB) 2105850.8

(51) Int. Cl. A23K 10/20; A23K 10/30; C12N 1/14; C12R 1/645

(52) U.S. Cl. CPC A23K 10/20; A23K 10/30; C12N 1/14; C12N 1/145; C12R 2001/645

(57) ABSTRACT

The present disclosure relates to a method for cultivation of insect biomass, for example, larvae and/or eggs using a feed comprising lignocellulose incubated with lignocellulose digesting fungal cells; a feed comprising pre-treated lignocellulose for cultivation of insect biomass; and a livestock feed obtained through the cultivation method of insect biomass.

### CROSS REFERENCE TO RELATED APPLICATIONS

This is the U.S. National Stage of International Application No. PCT/EP2022/060576, filed Apr. 21, 2022, which was published in English under PCT Article 21 (2), which in turn claims the benefit of GB Application No. 2105850.8, filed Apr. 23, 2021. The PCT application is herein incorporated by reference in its entirety.

### FIELD OF THE DISCLOSURE

The present disclosure relates to a method for preparation of insect biomass, for example, larvae and/or eggs, using a feed comprising lignocellulose incubated with lignocellulose digesting fungal cells; a feed comprising pre-treated lignocellulose for cultivation of insect biomass and a livestock feed comprising said insect biomass.

### Incorporation of Electronic Sequence Listing

The electronic sequence listing, submitted herewith as a txt file named sequence.txt (953 bytes), created on Oct. 16, 2023, is herein incorporated by reference in its entirety.

### Background to the Disclosure

The United Nations predicts that the human population will reach 9.8 billion by 2050 [1]. In less than three decades an additional 2 billion people will require sources of high-quality food resulting in a global food security challenge. Rising demand for sources of animal-derived protein such as fish, meat and eggs are an area of particular concern; a deficit of at least 60 Mt is projected by 2050, equivalent to an increase of 32% relative to current consumption [2]. Historically, per capita meat consumption has doubled since 1961 [3] and per capita fish consumption has increased from 9 kg in 1961 to 20.5 kg in 2018 (an increase of 128%) [4]. This has resulted in aquaculture becoming the fastest growing food production industry globally, to meet ever-growing demand. However, the largest caveat of animal protein production is that the animals are reliant upon protein sources such as soybean meal and fishmeal in their feed. Therefore, ever-increasing demand for animal-based protein will stimulate demand for these feed sources that are also diminishing in supply.

A replacement protein source is insect larvae, for example, Black Soldier Fly larvae (BSFL). BSFL are ideal to farm as they exhibit exceptional growth rates (increasing in weight by up to a factor of 10,000 in 14 days), high population densities and reproduction rates and suitability for vertical modular farming, which significantly reduces the land area required for their production and minimises scale-up risks. Their innate saprophytic ability enables them to metabolise a wide range of organic residue streams in nature, which when farmed facilitates the transformation of food wastes and residues into high quality protein sources. Importantly, BSFL have a protein content comparable to soybean meal and fishmeal (40-44%, dry weight) and an essential amino acid profile suitable for monogastric livestock and fish. This enables the direct replacement or substitution of soybean meal and fishmeal with BSFL meal in feed formulations. However, BSFL are typically reared on pre-consumer food waste. These wastes are limited to fruit and vegetable manufacturing/processing arisings that have not been in contact with meat from ruminant sources. This approach is not sustainable and has considerable food security implications. Furthermore, feedstock limitations prevent optimisation and homogeneity which restrict BSFL yields and yield reproducibility. These present circumstances of currently applicable feedstocks limit the total achievable BSFL production such that current and future demand is unlikely to be met. Moreover, pre-consumer food waste can comprise contaminants which should be excluded from the food chain.

Lignocellulose, the woody component of plant material, is a hugely abundant agricultural waste. Utilisation of lignocellulose however remains a challenge, as the extraction of fermentable sugars requires intensive physico-chemical pre-treatments and high loadings of enzyme cocktails. A key factor of this recalcitrance to degradation is the presence of lignin, a heterogeneous, hydrophobic aromatic polymer that encases the cellulose and hemicellulose, blocking enzyme accessibility and impeding cellulase activity. Despite extensive research into the biological degradation of lignocellulose and the mining of microbial communities for their ability to break down cellulose and hemicelluloses, lignocellulose has currently limited industrial use.

There is, therefore, a desire to identify lignocellulose digesting fungal species that efficiently convert lignocellulose.

Historically, wood-decaying fungi have been divided into white-, brown-, and soft-rots, depending on the morphology of their decomposition products. White-rot Basidiomycetous fungi, such as Ceriporiopsis subvermispora or Phanerochaete chrysosporium, are known for their ability to degrade and mineralize dark-coloured lignin and selectively enrich white cellulose. Although lignin modification has been recognized in brown-rot species, significant solubilization does not occur. Instead, the degradative strategy is typified by the selective removal of the polysaccharide components from the plant cell wall through chemical and enzymatic means.

Soft-rot fungi such as Ascomycetes deploy an alternative strategy. These fungi are capable of degrading lignocellulose through the secretion of large quantities of enzymes close to the site of attack due to the penetrating nature of their filamentous hyphae [5]. This causes characteristic softening of the lignocellulose, as the plant cell walls lose their structural integrity. However, Ascomycetes are not well known for their ability to solubilize lignin and, although there have been reports that they possess the capacity to modify and degrade lignin [6], it is not yet clear how this occurs.

Raw lignocellulosic biomass cannot support BSFL growth and no technology currently exists to economically convert the lignocellulosic biomass into high quality BSFL protein.

This disclosure relates to the identification of fungal species that efficiently degrade lignocellulose. An example of a lignocellulose degrading fungus is Parascedosporium putredinis NO1 which converts lignocellulose into a feed suitable for cultivating insect larvae. The converted lignocellulose allows rearing of the insect biomass to industrial standards and provides an animal feed which is free from contaminants and of consistent and high quality.

Parascedosporium putredinis NO1 is deposited under the Budapest Treaty on 29 Mar. 2022 with the International Depositary Authority, CABI BIOSCIENCE, UK Centre (IMI), Bakeham Lane, Egham, Surrey, United Kingdom, TW20 9TY and has been assigned accession number IMI 507228.

### STATEMENTS OF THE INVENTION

According to an aspect of the invention there is provided a method for the preparation of insect biomass comprising the steps:
i) forming composition comprising at least one lignocellulose digesting fungal cell and/or spore and at least one lignocellulose substrate;
ii) incubating the composition of i) to obtain a digested or partially digested lignocellulose substrate to form a pre-treated lignocellulose substrate; and
iii) contacting the pre-treated lignocellulose substrate with at least one insect species egg or larvae to provide insect biomass; and optionally,
iv) harvesting said insect biomass.

The five-stage life cycle of an insect is known and includes egg, larvae, prepupae, pupae and adult. The larvae and/or prepupae incubated and grown on the pre-treated lignocellulose substrate are in the context of this invention harvested shortly prior to reaching the pupae stage. “Insect biomass” comprises in the context of this invention the insect developmental stages from egg to larvae. The skilled person will acknowledge that the insect biomass can also comprise at a low ratio prepupae, however, development stages with high chitin content (e.g. the black soldier fly prepupae stage) should be avoided. Neonates are defined as egg-hatched larvae.

The term “fungal cell” includes hyphae and mycelium. Hyphae are the main mode of vegetative growth and are collectively called a mycelium.

The method steps i)-iii) can be temporally separated or performed simultaneously.

In a preferred method of the invention said lignocellulose substrate is particulate.

In a preferred method of the invention said method comprises the lyophilisation of the pre-treated lignocellulose substrate.

In an alternative preferred method of the invention said method comprises freezing of the pre-treated lignocellulose substrate.

The lignocellulose substrate can optionally be sterilised by heat such as for example for 20 min at 1.5 bar and 121° C. If the substrate is lyophilised it can be conveniently stored at room temperature.

In a preferred method of the invention said pre-treated lignocellulose substrate is adjusted to a moisture content of at least 40 w/v %, preferably between 40-95 w/v %.

In a further preferred method of the invention said pre-treated lignocellulose substrate is adjusted to a moisture content of between 50-85 w/v %, and preferably between 60-75 w/v %.

In a preferred method of the invention said lyophilised or frozen pre-treated lignocellulose substrate is adjusted to a moisture content of at least 40 w/v %, preferably between 40-95 w/v %.

In a further preferred method of the invention said lyophilised or frozen pre-treated lignocellulose substrate is adjusted to a moisture content of between 50-85 w/v %, and preferably between 60-75 w/v %

In a preferred method of the invention said insect biomass is subject to cold treatment.

Cold treatment is typically by chilling and storage at −20° C., or alternatively at between −10 to −20° C.

In a preferred method of the invention said insect biomass is separated from said pre-treated lignocellulose substrate and milled to form an animal feed.

Alternatively, said insect biomass is combined with pre-treated lignocellulose substrate and milled to form a composite animal feed.

In a preferred method of the invention said fungal cell or spore is from the family of Microascaceae.

In a preferred method of the invention said fungal cell or spore is an Ascomycete fungal species. In a preferred method of the invention said Ascomycete species is Parascedosporium sp.

In a preferred method of the invention said Parascedosporium sp. is Parascedosporium putredinis.

In a preferred method of the invention said Parascedosporium putredinis is Parascedosporium putredinis NO1.

In a preferred method of the invention said Parascedosporium putredinis NO1 is deposited under Accession number IMI 507228.

In a preferred method of the invention said lignocellulose substrate comprises lignocellulose obtained from the group consisting of: wheat straw, rice straw, corn stover, wheat bran, oat bran, oilseed rape straw, miscanthus straw, willow, sugarcane bagasse, oil palm empty fruit bunch and cacao husks.

In a preferred method of the invention said lignocellulose substrate comprises a composite of one or more selected from the group consisting of wheat straw, rice straw, corn stover, wheat bran, oat bran, oilseed rape straw, miscanthus straw, willow, sugarcane bagasse, oil palm empty fruit bunch and cacao husks.

In a preferred method of the invention said lignocellulose substrate is milled.

In a further preferred method of the invention said milled lignocellulose substrate has a particle size of between 0.1-15 mm.

In a preferred method of the invention said lignocellulose substrate is supplemented with fungal growth medium.

In a preferred method of the invention said lignocellulose is supplemented with fungal growth medium at between 1-50% w/v.

In a further preferred method of the invention said lignocellulose is supplemented with fungal growth medium at between 1-20% w/v.

In a further preferred method of the invention said lignocellulose is supplemented with fungal growth medium at between 20-50% w/v.

Growth medium for fungi is known in the art and provides typically a mix of nitrogen sources, inorganic salts, inorganic sulphur-containing compounds, phosphorus, chelating agents, vitamins and/or trace elements. Inorganic salt compounds which may be present in the media comprise the chloride, phosphorus and sulphate salts of calcium, magnesium, sodium, cobalt, molybdenum, potassium, manganese, zinc, copper, and iron. Inorganic sulphur-containing compounds such as, for example, sulphates, sulphites, dithionites, tetrathionates, thiosulfates, sulphides, or else organic sulphur compounds such as mercaptans and thiols, may be used as sources of sulphur for the production of sulphur-containing fine chemicals and pathway intermediates, in particular of methionine. Phosphoric acid, potassium dihydrogenphosphate or dipotassium hydrogenphosphate or the corresponding sodium-containing salts may be used as sources of phosphorus. Chelating agents may be added to the medium in order to keep the metal ions in solution. Particularly suitable chelating agents comprise dihydroxyphenols such as catechol or protocatechuate and organic acids such as citric acid. Typically, fungal growth medium includes nitrogen sources which are usually organic or inorganic nitrogen compounds or materials comprising these compounds. Examples of nitrogen sources comprise ammonia in liquid or gaseous form or ammonium salts such as ammonium sulphate, ammonium chloride, ammonium phosphate, ammonium carbonate or ammonium nitrate, nitrates, urea, amino acids, or complex nitrogen sources such as cornsteep liquor, soya meal, soya protein, yeast extract, meat extract, and others. The nitrogen sources can be used individually or as a mixture.

In a preferred method of the invention said fungal growth medium comprises or consists of yeast extract, KCl, KH₂PO₄, K₂HPO₄, MgSO₄, NaNO₃ and trace elements.

In a further preferred method of the invention said trace elements are selected from the group consisting of EDTA disodium salt, ZnSO₄, H₃BO₃, MnCl₂, CoCl₂, CuSO₄, (NH₄)6Mo₇O₂₄ and FeSO₄.

In a further preferred method of the invention said fungal growth medium consists of 8.55 g/L yeast extract, 0.52 g/L KCl, 0.815 g/L KH₂PO₄, 1.045 g/L K₂HPO₄, 1.35 g/L MgSO₄, 1.75 g/L NaNO₃ and trace elements consisting of 50 g/L Na₂EDTA·2H₂O, 22 g/L ZnSO₄·7H₂O, 11.4 g/L H₃BO₃, 0.506 g/L MnCl₂·4H₂O, 0.4499 g/L FeSO₄·7H₂O, 0.161 g/L CoCl₂·6H₂O, 0.157 g/L CuSO₄·5H₂O and 0.110 g/L (NH₄)₆Mo₇O₂₄·4H₂O.

In a preferred method of the invention said lignocellulose substrate is further supplemented with a nitrogen source.

In a preferred method of the invention said nitrogen source is provided at between 1-50% w/v fungal growth medium.

In a preferred method of the invention said nitrogen source is provided at between 1-35% w/v fungal growth medium.

In a preferred method of the invention said nitrogen source is provided at a carbon to nitrogen ratio of between 60:1 and 4:1.

In a preferred method of the invention said nitrogen source is provided at a carbon to nitrogen ratio of between 60:1 and 10:1.

In a further preferred method of the invention said nitrogen source is provided at a carbon to nitrogen ratio of between 50:1 and 20:1, and more preferably between 40:1 and 30:1.

In a further preferred method of the invention said nitrogen source is provided at a carbon to nitrogen ratio at 60:1, 50:1, 40:1, 30:1, 25:1, 20:1, 15:1, 10:1, 5:1, 4:1, 3:1 or 2:1.

The carbon to nitrogen ratio is typically analysed using an elemental analyser.

In a preferred method of the invention said nitrogen source is selected from the group consisting of wheat bran, peptone, oat bran, macro algae, pea by-products, maize bran, feather meal.

In a preferred method of the invention said insect species larvae are between 1-2 or 3-5 days old.

In a preferred method of the invention said insect species larvae are neonates.

In a further preferred method of the invention said insect species is selected from the group consisting of: house fly (Musca domestica), black soldier fly (Hermetia illucens), mealworms (Tenebrio molitor), locust (Locusta migratoria, Schistocerca gregaria, Oxya sp.), and silkworms (Bombyx mori).

In a further preferred method of the invention said insect species is selected from the group consisting of: house fly (Musca domestica), black soldier fly (Hermetia illucens), mealworms (Tenebrio molitor), locust (Locusta migratoria, Schistocerca gregaria, Oxya sp.), crickets (Acheta domesticus and Teleogryllus commodus), cockroach (Periplaneta americana) and silkworms (Bombyx mori).

In a further preferred method of the invention said insect species is black soldier fly egg.

In a further preferred method of the invention said insect species is black soldier fly larvae.

In a further preferred method of the invention said black soldier fly larvae are neonates.

In a further preferred method of the invention said insect biomass comprises insects selected from the group consisting of: house fly egg or larvae (Musca domestica), black soldier fly egg or larvae (Hermetia illucens), mealworms (Tenebrio molitor), locust egg or nymph (Locusta migratoria, Schistocerca gregaria, Oxya sp.), and silkworms (Bombyx mori).

In a further preferred method of the invention said insect biomass comprises insects selected from the group consisting of: house fly egg or larvae (Musca domestica), black soldier fly egg or larvae (Hermetia illucens), mealworms (Tenebrio molitor), locust egg or nymph (Locusta migratoria, Schistocerca gregaria, Oxya sp.), crickets (Acheta domesticus and Teleogryllus commodus), cockroach (Periplaneta americana) and silkworms (Bombyx mori).

In a further preferred method of the invention said insect biomass comprises house fly larvae (Musca domestica), black soldier fly larvae (Hermetia illucens), mealworms (Tenebrio molitor), locust nymph (Locusta migratoria, Schistocerca gregaria, Oxya sp.), crickets (Acheta domesticus and Teleogryllus commodus), cockroach (Periplaneta americana) and silkworms (Bombyx mori).

In a further preferred method of the invention said insect biomass comprises black soldier fly larvae.

In a preferred method of the invention said lignocellulose digesting fungus is provided at a concentration of between 2.86×10⁶ and 2.86×10¹² spores/kg lignocellulose substrate.

In a further preferred method of the invention said lignocellulose digesting fungus is provided at a concentration of between 2.86×10⁷ and 2.86×10¹¹ spores/kg lignocellulose substrate, and more preferably between 2.86×10⁸ and 2.86×10¹⁰ spores/kg lignocellulose substrate.

In a preferred method of the invention said lignocellulose digesting fungus is provided at a concentration of between 0.1-50 g mycelia/kg lignocellulose substrate.

In a preferred method of the invention said lignocellulose digesting fungus is provided at a concentration of between 0.1-10 g mycelia/kg lignocellulose substrate.

In a further preferred method of the invention said lignocellulose digesting fungus is provided at a concentration of between 0.5-5 g mycelia/kg lignocellulose substrate, and more preferably between 1-5 g mycelia/kg lignocellulose substrate.

In an alternative preferred method of the invention said lignocellulose digesting fungus is provided at a concentration of between 0.1-50 g mycelia/l fungal growth medium, and more preferably between 10-30 g mycelia/l fungal growth medium, and even more preferably between 5-10 g mycelia/l fungal growth medium

In an alternative preferred method of the invention said lignocellulose digesting fungus is provided at a concentration of between 1×10¹ and 1×10⁶ spores/mL fungal growth medium.

In a further alternative preferred method of the invention said lignocellulose digesting fungus spore is provided at a concentration of between 1×10² and 1×10⁵ spores/mL fungal growth medium.

In a further alternative preferred method of the invention said lignocellulose digesting fungus is provided at a concentration of between 1×10³ and 1×10⁴ spores/mL fungal growth medium.

In a preferred method of the invention said lignocellulose digesting fungus is incubated with the lignocellulose substrate under aerobic conditions at a temperature of between 20° C. and 35° C. for 3-28 days.

In a preferred method of the invention said lignocellulose digesting fungus is incubated with the lignocellulose substrate under aerobic conditions at a temperature of between 20° C. and 35° C. for 10-20 days.

Aerobic conditions can be ensured by agitation and/or rotation of the material.

In a preferred method of the invention said pre-treated lignocellulose substrate is provided to the insect species egg or larvae at a feeding rate of between 50 and 200 mg per larva per day.

In a preferred method of the invention said pre-treated lignocellulose substrate is provided to the insect biomass at a feeding rate of between 50 and 200 mg per larva per day.

In a further preferred method of the invention said feeding rate is between 75-150 mg per larva per day.

In a further preferred method of the invention said feeding rate is 30-40 mg per larva per day.

In a further preferred method of the invention said feeding rate is 100 mg per larva per day.

In a preferred method of the invention said pre-treated lignocellulose substrate is incubated with insect egg or larvae for between 7-21 days.

In a preferred method of the invention said pre-treated lignocellulose substrate is incubated with insect larvae for between 8-16 days.

In a preferred method of the invention said pre-treated lignocellulose substrate is incubated with insect egg for between 8-16 days.

In a further preferred method of the invention said pre-treated lignocellulose substrate is incubated with insect egg or larvae for between 10-14 days.

In a preferred method of the invention said pre-treated lignocellulose substrate is incubated at a temperature of between 25° C. and 30° C.

In a preferred method of the invention said pre-treated lignocellulose substrate is incubated at a relative humidity of between 60-80%; preferably between 60 and 70% relative humidity.

In a preferred method of the invention said method yields an increase in larval weight of between 60-200 mg per larva in between 7-20 days. Preferably, said method yields an increase in larval weight of between 60-120 mg per larva in between 7-20 days.

In a further preferred method of the invention said method yields an increase in larval weight of between 80-100 mg per larva in between 10-14 days.

According to a further aspect of the invention there is provided an insect feed obtained by the method according to the invention.

According to an aspect of the invention there is provided an animal feed comprising insect biomass obtained by the method according to the invention.

According to an aspect of the invention there is provided a lignocellulose digesting fungal strain Parascedosporium putredinis NO1 deposited under accession number IMI 507228.

Parascedosporium putredinis NO1 is deposited under the Budapest Treaty on 29 Mar. 2022 with the International Depositary Authority, CABI BIOSCIENCE, UK Centre (IMI), Bakeham Lane, Egham, Surrey, United Kingdom, TW20 9TY and has been assigned accession number IMI 507228.

According to an aspect of the invention there is provided a lignocellulose digesting fungal cell wherein said fungal cell is:
i) a fungal cell comprising a nucleic acid sequence that hybridises under high stringency conditions with a nucleic acid probe complementary to the nucleotide sequence set forth in SEQ ID NO: 1; or
ii) a fungal cell comprising a nucleotide sequence as set forth in SEQ ID NO: 1 that has 95%, 96%, 97%, 98% or 99% nucleotide sequence identity to the full-length sequence set forth in SEQ ID NO: 1.

In a preferred embodiment of the invention said lignocellulose digesting fungal cell comprises a nucleic acid molecule comprising the nucleotide sequence as set forth in SEQ ID NO: 1, or polymorphic sequence variants of SEQ ID NO: 1.

In a preferred embodiment of the invention said fungal cell is an Ascomycete fungal species.

In a preferred embodiment of the invention said Ascomycete species is Parascedosporium sp.

In a preferred embodiment of the invention said Parascedosporium sp. is Parascedosporium putredinis

In a preferred embodiment of the invention said Parascedosporium putredinis is Parascedosporium putredinis NO1 deposited under accession number IMI 507228.

According to an aspect of the invention there is provided a cell culture comprising cells of the fungal strain Parascedosporium putredinis NO1 deposited under accession number IMI 507228 wherein said culture is substantially a monoculture of said fungal strain.

A monoculture in the context of this invention is a pure culture comprising Parascedosporium putredinis NO1 [IMI 507228] and medium for maintenance or growth of the fungus.

According to a further aspect of the invention there is provided a lignocellulose digesting fungal cell wherein said cell is characterised by at least one amplicon wherein said at least one amplicon is amplified by a polymerase chain reaction wherein said reaction comprises oligonucleotide primer pairs comprising or consisting of:

| (SEQ ID NO: 2) | |
|---|---|
| | TCCGTAGGTGAACCTGCGG |
| | and |
| (SEQ ID NO: 3) | |
| | CGCTGCGTTCTTCATCG. |

In a preferred embodiment of the invention said fungal cell is an Ascomycete fungal species.

In a preferred embodiment of the invention said Ascomycete species is Parascedosporium sp.

In a preferred embodiment of the invention said Parascedosporium sp. is Parascedosporium putredinis

In a preferred embodiment of the invention said Parascedosporium putredinis is Parascedosporium putredinis NO1 deposited under accession number IMI 507228.

According to an aspect of the invention there is provided the use of a lignocellulose digesting fungal cell in a method for the preparation of insect biomass wherein said fungal cell is:
i) a fungal cell comprising a nucleic acid sequence that hybridises under high stringency conditions with a nucleic acid probe complementary to the nucleotide sequence set forth in SEQ ID NO: 1; or
ii) a fungal cell comprising a nucleotide sequence as set forth in SEQ ID NO: 1 that has 95%, 96%, 97%, 98% or 99% nucleotide sequence identity to the full-length sequence set forth in SEQ ID NO: 1.

In a preferred embodiment of the invention said fungal cell is an Ascomycete fungal species.

In a preferred embodiment of the invention said Ascomycete species is Parascedosporium sp.

In a preferred embodiment of the invention Parascedosporium sp. is Parascedosporium putredinis

In a preferred embodiment of the invention said fungal cell is Parascedosporium putredinis NO1 deposited under accession number IMI 507228.

According to an aspect of the invention there is provided the use of a lignocellulose digesting fungal cell in a method for the preparation of animal feed wherein said fungal cell is:
i) a fungal cell comprising a nucleic acid sequence that hybridizes under high stringency conditions with a nucleic acid probe complementary to the nucleotide sequence set forth in SEQ ID NO: 1; or
ii) a fungal cell comprising a nucleotide sequence as set forth in SEQ ID NO: 1 that has 95%, 96%, 97%, 98% or 99% nucleotide sequence identity to the full-length sequence set forth in SEQ ID NO: 1.

In a preferred embodiment of the invention said lignocellulose digesting fungal cell comprises a nucleic acid molecule comprising the nucleotide sequence as set forth in SEQ ID NO: 1, or polymorphic sequence variants of SEQ ID NO: 1.

Hybridisation of a nucleic acid molecule occurs when two complementary nucleic acid molecules undergo an amount of hydrogen bonding to each other. The stringency of hybridisation can vary according to the environmental conditions surrounding the nucleic acids, the nature of the hybridisation method, and the composition and length of the nucleic acid molecules used. Calculations regarding hybridisation conditions required for attaining particular degrees of stringency are discussed in Sambrook et al., Molecular Cloning: A Laboratory Manual (Cold Spring Harbor Laboratory Press, Cold Spring Harbor, N Y, 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridised to its complementary strand. The following is an exemplary set of hybridisation conditions and is not limiting:

Very High Stringency (Allows Sequences that Share at Least 90% Identity to Hybridise)
Hybridisation: 5×SSC at 65° C. for 16 hours
Wash twice: 2×SSC at room temperature (RT) for 15 minutes each
Wash twice: 0.5×SSC at 65° C. for 20 minutes each
   High Stringency (Allows Sequences that Share at Least 80% Identity to Hybridise)
Hybridisation: 5×-6×SSC at 65° C.-70° C. for 16-20 hours
Wash twice: 2×SSC at RT for 5-20 minutes each
Wash twice: 1×SSC at 55° C.-70° C. for 30 minutes each

In a preferred embodiment of the invention said fungal cell is an Ascomycete fungal species.

In a preferred embodiment of the invention said Ascomycete species is Parascedosporium sp.

In a preferred embodiment of the invention Parascedosporium sp. is Parascedosporium putredinis

In a preferred embodiment of the invention said Parascedosporium putredinis is Parascedosporium putredinis NO1 deposited under accession number IMI 507228.

According to a further aspect of the invention there is provided the use of a lignocellulose digesting fungal cell in a method for the preparation of insect biomass wherein said cell is characterised by at least one amplicon wherein said at least one amplicon is amplified by a polymerase chain reaction wherein said reaction comprises oligonucleotide primer pairs comprising or consisting of:

| (SEQ ID NO: 2) | |
|---|---|
| | TCCGTAGGTGAACCTGCGG |
| | and |
| (SEQ ID NO: 3) | |
| | CGCTGCGTTCTTCATCG. |

In a preferred embodiment of the invention said fungal cell is an Ascomycete fungal species.

In a preferred embodiment of the invention said Ascomycete species is Parascedosporium sp.

In a preferred embodiment of the invention Parascedosporium sp. is Parascedosporium putredinis

In a preferred embodiment of the invention said Parascedosporium putredinis is Parascedosporium putredinis NO1 deposited under accession number IMI 507228.

According to a further aspect of the invention there is provided the use of a lignocellulose digesting fungal cell in a method for the preparation of animal feed wherein said cell is characterised by at least one amplicon wherein said at least one amplicon is amplified by a polymerase chain reaction wherein said reaction comprises oligonucleotide primer pairs comprising or consisting of:

| (SEQ ID NO: 2) | |
|---|---|
| | TCCGTAGGTGAACCTGCGG |
| | and |
| (SEQ ID NO: 3) | |
| | CGCTGCGTTCTTCATCG. |

In a preferred embodiment of the invention said fungal cell is an Ascomycete fungal species.

In a preferred embodiment of the invention said Ascomycete species is Parascedosporium sp.

In a preferred embodiment of the invention Parascedosporium sp. is Parascedosporium putredinis.

Preferably, said Parascedosporium putredinis is Parascedosporium putredinis NO1 deposited under accession number IMI 507228.

According to an aspect of the invention there is provided a bioreactor comprising: at least one lignocellulose digesting fungal cell and/or spore and at least one lignocellulose substrate; and at least one insect species egg or larva to provide insect biomass.

In a preferred embodiment of the invention said fungal cell is an Ascomycete fungal species.

In a preferred embodiment of the invention said Ascomycete species is Parascedosporium sp.

In a preferred embodiment of the invention Parascedosporium sp. is Parascedosporium putredinis.

Preferably, said Parascedosporium putredinis is Parascedosporium putredinis NO1 deposited under accession number IMI 507228.

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with an aspect, embodiment or example of the invention are to be understood to be applicable to any other aspect, embodiment or example described herein unless incompatible therewith.

An embodiment of the invention will now be described by example only and with reference to the following figures:

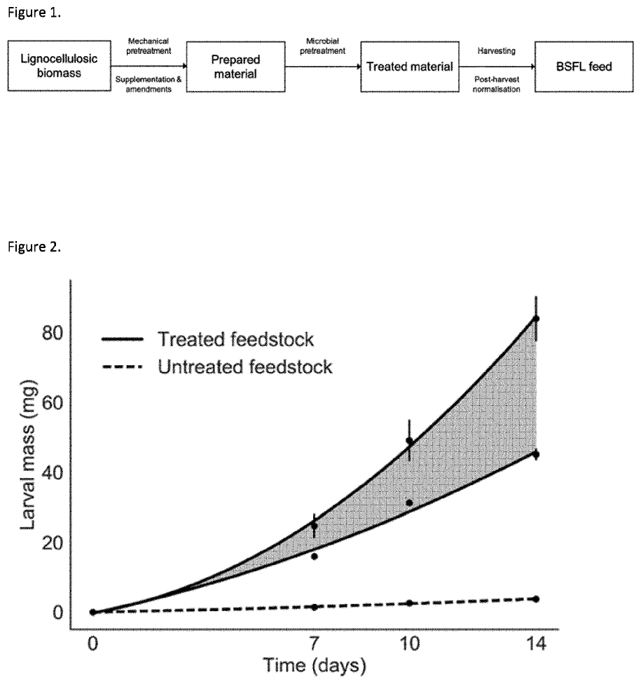

FIG. 1. Flow chart of the method to convert lignocellulosic biomass (and other agricultural residues) into BSFL feed.

FIG. 2. Growth curves for BSFL fed with untreated lignocellulosic material (‘untreated feedstock’; wheat straw) and treated material (‘treated feedstock’; pre-treated wheat straw). Data shows change in mean mass [mg] per larva (± standard deviation) over time [days], based on the average of 10 randomly selected larvae per biological replicate (n=3). The greyed-out area between the two ‘treated feedstock’ curves indicates a larval performance range dependent upon treatment conditions. All larvae received 100 mg feed/larva/day.

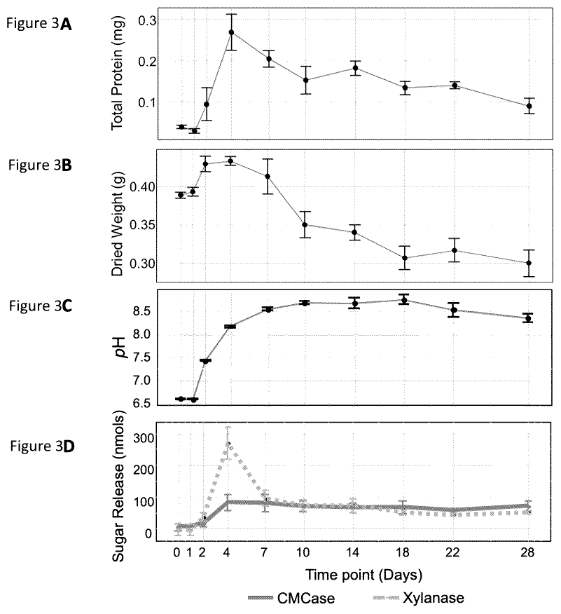

FIGS. 3A-3D. Growth of P. putredinis NO1 on wheat straw over a period of one month. a) Growth of P. putredinis NO1 on wheat straw estimated by the total protein present in the culture. b) The dried weight of the total biomass within the culture. c) The pH of the culture was also monitored alongside. d) The release of sugar after 1 h from 10% supernatant loading on carboxymethylcellulose and beechwood xylan.

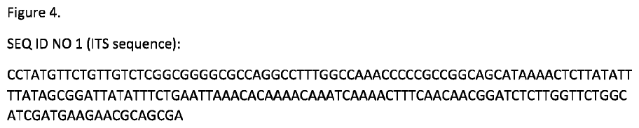

FIG. 4: ITS sequence (SEQ ID NO 1).

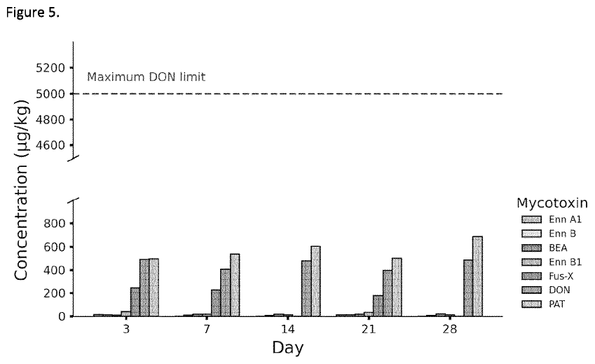

FIG. 5: Mycotoxin testing conducted on wheat straw treated with P. putredinis NO1 detected 7 mycotoxins across all samples, with all those regulated under FSA animal feed legislation significantly below limits. LC-MS/MS based analysis of 68 mycotoxins using solvent calibration standards for analyte identity and quantification. Enn A1=enniatin A1, Enn B=enniatin B, BEA=beauvericin, Enn B1=enniatin B1, Fus-X=fusarenon X, DON=deoxynivalenol, PAT=patulin. Dotted line indicates the maximum DON limit tolerated by animal feed legislation (5000 μg/kg).

### MATERIAL AND METHODS

Wheat Straw Degradation in Shake-Flasks Inoculated with Compost

Two-liter shake flasks, containing 1 L minimal media and 5% (w/v) milled wheat straw, were inoculated with 1% (w/v) compost. The inoculum was collected from composting wheat straw that had been developed over the period of a year and watered at regular intervals. The inoculum was prepared by blending until homogenised and used on the day of preparation. The minimal media was based on Aspergillus niger minimal media and contained KCl 0.52 g/L, KH₂PO₄ 0.815 g/L, K₂HPO₄ 1.045 g/L, MgSO₄ 1.35 g/L, NaNO₃ 1.75 g/L and Hutner's trace elements (Na₂EDTA·2H₂O 50 g/L, ZnSO₄·7H₂O 22 g/L, H₃BO₃ 11.4 g/L, MnCl₂·4H₂O 0.506 g/L, FeSO₄·7H₂O 0.4499 g/L, CoCl₂·6H₂O 0.161 g/L, CuSO₄·5H₂O 0.157 g/L, (NH₄)₆Mo₇O₂₄·4H₂O 0.110 g/L). These flasks were incubated at 30° C. and shaken at 150 rpm. Aliquots (10 mL) containing both the solid and liquid fractions were aseptically collected weekly for eight weeks. The samples were then serially diluted with ×1 phosphate-buffered saline to concentrations ranging between 10⁻¹ and 10⁻⁷. From these dilutions 100 μL samples were used to create spread plates on both nutrient agar (NA) and potato dextrose agar (PDA), in order to culture strains from the composting environment.

### Targeted Amplicon Sequencing of 18S and ITS Region

Genomic DNA was harvested from the compost cultures using a modified CTAB protocol adapted for use on materials with high phenolic contents. From the composting shake flask, 20 mL aliquots were harvested weekly. The biomass was separated from the liquid fraction by centrifugation performed at 4000 g at 4° C., and 0.5 g of biomass removed to a 2 mL screw-cap tube. To this 500 μL of cetyltrimethylammonium bromide (CTAB) buffer (2% (w/v) CTAB 100 mM Tris-HCl (pH 8.0), 20 mM EDTA (pH 8.0), 2 M NaCl, 2% (w/v) polyvinylpyrrolidone (Mr 40.000), 5% 2-mercaptoethanol (v/v), 10 mM ammonium acetate, was added along with 0.5 g of zirconia beads and 0.5 mL of phenol:chloroform:isoamyl alcohol (25:24:1, pH 8.0), before briefly vortexing. The material was then bead-beaten using a TissueLyser II (Qiagen) for 5 min at speed 28/s. A modified phenol-chloroform method was used to extract DNA after cell lysis. The sample was spun for 5 min. at max. speed to achieve separation of the phases before the aqueous layer was removed to a fresh 2 mL Eppendorf tube. To the aqueous phase chloroform:isoamyl alcohol (21:1) was added, and this was spun and the aqueous phase transferred to a fresh tube, to remove any remaining phenolics. To precipitate the DNA within the sample, an equal volume of ice-cold 100% isopropanol was added and incubated for 1 h. DNA was pelleted by centrifugation at 13,000 rpm for 10 min, and supernatant was removed without disturbing the pellet. The pellet was then washed with 80% ethanol, before being re-suspended in DNAse-free water.

Regions for amplicon sequencing were amplified using Phusion® High-Fidelity DNA Polymerase (Finnzymes OY, Finland) as per manufactures instructions before being purified with Agencourt AMPure XP (Beckman Coulter) and sequenced at the Biorenewables Development Centre (BDC), York, U.K. using an Ion Torrent platform. The primers pairs, for ITS, were as follows; ITS1 Fw-TCCGTAGGTGAACCTGCGG (SEQ ID NO: 2), Rv-CGCTGCGTTCTTCATCG (SEQ ID NO: 3).). Ribosomal DNA sequence data generated via targeted amplicon sequencing was analysed using the open-access software Qiime on the University of York's Technology Facilities linux server⁷ Each fastq file generated from the IonTorrent platform was first demultiplexed and then converted into both fasta and qual file types using Qiimes python script convert_fastaqual_fastq.py. To remove the primer sequences from the reads, the script split_libraries.py was used along with a mapping file generated as per Qiimes instructions. Low-quality reads were removed by filtering out reads under 180 bp long and those without recognisable primers. The orientation of the sequences was then corrected based on the primer location. Operational taxonomic units (OTUs) were then created from the fasta files. These files were picked using the open-reference OTU picking process. To perform this, the script pick_open_reference_otus.py was used. This step also includes taxonomy assignment, sequence alignment, and tree building steps. For the taxonomy assignments of the fungal ITS sequences the UNITE (alpha release 12_11) database was used.⁸

### Central Composite Design for Media Optimisation

The optimised media for Parascedosporium putredinis NO1 growth consisted of yeast extract 8.55 g/L, KCl 0.52 g/L, KH₂PO₄ 0.815 g/L, K₂HPO₄ 1.045 g/L, MgSO₄ 1.35 g/L, NaNO₃ 1.75 g/L and Hutner's trace elements.

### Characterisation of Parascedosporium Putredinis NO1 Growth on Wheat Straw

Growth of Parascedosporium putredinis NO1 was assessed a range of approaches (FIGS. 3A-3D), such as using the dried weight of the biomass present within the culture. Cultures were transferred to pre-weighed and freeze-dried falcon tubes and chilled for 5 min. They were then centrifuged at 4,500 rpm, and the supernatant removed. The biomass was gently rinsed with ×1 PBS and tubes were flash-frozen in liquid nitrogen and lyophilised. Each tube was then re-weighed to calculate the dry weight of the biomass present. The total protein content of the cultures was used as an indicator of growth on insoluble materials such as wheat straw. Total protein was extracted by boiling 100 μg of freeze-dried biomass in 1 mL of 0.2% (w/v) sodium dodecyl sulfate, for 5 min. to lyse all cells present. Protein was then collected by centrifugation at 14,000 rpm and the supernatant collected into a fresh 50 mL falcon tube. This was repeated three times, without heating, and with vigorous vortexing between each centrifuge step to wash the biomass of any remaining protein. Extracted protein was precipitated with five volumes of ice-cold acetone overnight at −20° C., before being centrifuged at 4500 rpm and the resulting pellet washed with 80% (v/v) ice-cold ethanol. The ethanol-protein mix was then centrifuged again, and the supernatant removed and the pellet air-dried. The protein was then solubilised in 3 mL of H₂O and quantified using the Bradford assay. The ability of an enzyme to cleave polysaccharides and produce products with reducing ends was assessed at each timepoint by incubating 10 μL of cultural supernatant with the 2% (w/v) of either carboxymethylcellulose (CMC) or xylan (beechwood) in 200 μL of 50 mM sodium phosphate at 6.8 and 30° C. Before and after incubation 10 μL aliquots mixed with p-hydroxybenzoic acid hydrazide (PAHBAH), heated to 70° C. for 10 min, and color change detected at 415 nm using a microtitre Tecan Safire2 plate reader.⁶² A stock solution of the appropriate monosaccharide was assayed to obtain a standard curve for quantification of sugar release.

Parascedosporium putredinis NO1 Spore Preparation and Quantification

For the preparation of P. putredinis NO1 spores, potato glucose agar slants are inoculated with P. putredinis NO1 by streaking from an existing stock and incubated at 30° C. until sporulation. After approximately 14 days, spores are harvested into sterile saline solution (0.5% NaCl w/v, 0.2% Tween 20 v/v; 500 μL per agar slant) and combined into a single suspension for quantification. For fungal spore quantification, a dilution (e.g. 1/10, 1/100) of spore suspension is made up and 10 μL applied to both counting chambers of a clean haemocytometer (Improved Neubauer 1/400 mm, Hawksley) with coverslip. Spores are counted under ×40 magnification and the following calculations are used to quantify the number of spores in the original suspension (N=number of spores counted within each haemocytometer grid).

$$Number of spores per mL of diluted suspension ( A ) = ( N / 8 ) \times {10}^{4}$$

$$Number of spores per mL of original suspension = A \times dilution factor ( e . g \text{.10} , 100 )$$

Spore solution is stored at 4° C., with glycerol stocks kept at −20° C. (glycerol=30% v/v).

Treatment of Wheat Straw with Parascedosporium Putredinis NO1 Under Solid State Fermentation (SSF) Conditions

Lignocellulosic material (e.g. wheat straw, rice straw, corn stover, wheat bran, oat bran, oilseed rape straw, miscanthus straw, willow, sugarcane bagasse, oil palm empty fruit bunch, cacao husks) is converted into BSFL feed according to FIG. 1. Initially, biomass is prepared through milling and grading (particle size range=100 μm-15 mm, inclusive). Milled biomass is supplemented (solid-state=20% to 50% w/v, inclusive; liquid-state=1% to 20% w/v, inclusive) with P. putredinis NO1 growth medium (yeast extract-8.55 g/L, KCl-0.52 g/L, KH₂PO₄-0.815 g/L, K₂HPO₄-1.045 g/L, MgSO₄-1.35 g/L, NaNO₃-1.75 g/L) and Hutner's trace elements (containing EDTA disodium salt, ZnSO₄·7H2O, H₃BO₃, MnCl₂·4H₂O, CoCl₂·6H₂O, CuSO₄·5H₂O, (NH₄)₆Mo₇O₂₄·4H₂O, FeSO₄·7H₂O, 1/1000 dilution). The supplemented product is amended further with wheat bran (1-35% w/v, or other material) to a carbon/nitrogen ratio of between 60:1 and 10:1 or between 60:1 and 4:1 to form “prepared material”. The prepared material is sterilised at 121° C. for 15 min and inoculated with P. putredinis NO1 using spores at a concentration of between 2.86×10⁶ and 2.86×10¹² spores/kg biomass, or mycelia (by mass). Vessels containing prepared material inoculated with P. putredinis NO1 are incubated at a temperature of between 20° C. and 35° C. with or without agitation/rotation for 3-28 days, with routine homogenisation to ensure maintenance of aerobic conditions. This produces “treated material” with evidence of mass loss as a result of P. putredinis NO1's action. Treated material is then harvested and optionally lyophilized for storage at room temperature, or alternatively frozen. If being fed to BSFL, treated material is subjected to post harvest normalisation, conducted by calculation of moisture content and adjustment through the addition of water (or other liquid diluent) to between 40% and 95% (w/v).

### Mycotoxin Testing

Mycotoxin testing is conducted on treated material to ensure feed safety standards. LC-MS/MS based analysis is used to screen for 68 mycotoxins (including all those regulated under FSA animal feed legislation) using solvent calibration standards for analyte identity and quantification. Analysis is conducted by Fera Science Ltd. (UK).

### Larval Rearing

BSFL (either neonate or 1-5 days old) are fed treated material using a feeding rate of between 50 and 200 mg/larva/day (wet weight) for between 8 to 16 days, at a temperature of between 25° C. and 30° C. and relative humidity between 60% and 80%. BSFL are then separated from the frass, harvested and “devitalised” by storage at −20° C.

### EXAMPLE 1

Isolation of Parascedosporium putredinis NO1

We inoculated liquid cultures containing wheat straw as the sole carbon source with samples of wheat straw-enriched compost and tracked the dynamics of the resulting microbial community using targeted amplicon sequencing during cultivation. Sequencing of 16S ribosomal RNA genes generated over three million reads from the prokaryotic community over the whole time course, which clustered together to form 25,304 operational taxonomic units (OTUs). The most abundant bacterial phyla identified were the gram-negative Bacteroidetes, Verrucomicrobia and Proteobacteria, respectively, representing an average of 31%, 19.8%, and 15.5% of the total reads across the time course. Analysis of the eukaryotic community by sequencing the Internal Transcribed Spacer (ITS) region predominantly yielded reads that had no match within the UNITE fungal rDNA sequence database.^{9,10} In total, 96.5% of generated OTUs were not recognised as fungal and instead showed the closest homologies to protozoa. Among the fungi, we noted distinct changes in the composition of the community with time. In particular, a fungus (designated strain NO1) identified as Parascedosporium putredinis (an Ascomycete in the Microascaceae family), showed increased abundance after four weeks of incubation. This fungus was readily isolated from shake flasks by culturing on both nutrient agar and potato dextrose agar and dominated the eukaryotic community in the shake flasks after four weeks of incubation, representing 84% of the identifiable fungal reads at eight weeks, a time point by which, we hypothesise, the majority of easily accessible carbon from wheat straw has been depleted.¹¹ Interestingly, this fungus could be selectively cultivated when agar plates contained kraft lignin as the sole carbon source.

### EXAMPLE 2

BSFL feeding trials (14 days) are set up in biological triplicate using 50 neonate (egg-hatched) larvae per replicate. Larvae are fed using a daily feeding rate of 100 mg/larva (wet weight) in accordance with previous research [12]. To do this, feed (i.e. P. putredinis NO1 treated material) is added to the feeding pots every 3-4 days (or other frequency), beginning at day 0. To monitor larval growth rates over time, groups of ten larvae are selected randomly at various time points (days 7, 10 and 14), weighed using a fine analytical balance and then returned (i.e. non-destructive sampling). FIG. 2 shows growth curves for BSFL fed with untreated lignocellulosic material (‘untreated feedstock’; wheat straw) and treated material (‘treated feedstock’; pre-treated wheat straw) using this methodology. At the end of each feeding trial, surviving larvae are counted for calculation of survival rate and weighed for calculation of total mass. Where applicable, the number of pre-pupal larvae is also noted. Results are graphically represented using self-written R or Python scripts. Positive controls are set up alongside, each using 50 neonate larvae per replicate, fed on chick starter crumb (nutritionally optimised diet, ˜68% moisture content) at a daily feeding rate of 37.26 mg/larva (wet weight) to correct for the differences in moisture contents between test and positive control feeds.

### EXAMPLE 3

Mycotoxin testing (FIG. 5) conducted on wheat straw treated with P. putredinis NO1. The concentration of P. putredinis NO1 was 2.86×10⁶ spores/kg biomass (wheat straw). LC-MS/MS based analysis using solvent calibration standards for analyte identity and quantification of 68 mycotoxins-including those regulated by the FSA under animal feed legislation. 7 detectable mycotoxins across all samples, out of 68 mycotoxins tested for. Deoxynivalenol (DON) max. limit in animal feed=5000 μg/kg. Levels found in all samples well below limit.

### REFERENCES

[1] DESA U. World Population Prospects 2019. United Nations. Department of Economic and Social Affairs. World Population Prospects 2019.
[2] Henchion et al. Foods 2017, 6:21.
[3] Ritchie H: Meat and Dairy Production. Our World in Data 2017.
[4] FAO: The State of World Fisheries and Aquaculture, 2000. Agriculture Organization of the United Nations. Fisheries Department. 2000.
[5] F. Barras, F. Van Gijsegem, A. K. Chatterjee, Extracellular enzymes and pathogenesis of soft-rot Erwinia. Annu. Rev. Phytopathol. 32, 201-234 (2003).
[6]. G. van Erven et al., Evidence for ligninolytic activity of the ascomycete fungus Podospora anserina. Biotechnol. Biofuels 13, 75 (2020).
[7]. Caporaso J G, et al. QIIME allows analysis of high-throughput community sequencing data. Nature methods 7, 335-336 (2010).
[8]. Abarenkov K, et al. The UNITE database for molecular identification of fungi-recent updates and future perspectives. The New phytologist 186, 281-285 (2010).
[9] Kõljalg U, et al. UNITE: a database providing web-based methods for the molecular identification of ectomycorrhizal fungi. 166, 1063-1068 (2005).
[10] Abarenkov K, et al. The UNITE database for molecular identification of fungi-recent updates and future perspectives. 186, 281-285 (2010).
[11] Alessi A M, et al. Defining functional diversity for lignocellulose degradation in a microbial community using multi-omics studies. Biotechnol Biofuels 11, 166 (2018).
[12] Stefan Diener, et al., 2009, Conversion of organic material by black soldier fly larvae: establishing optimal feeding rates. Waste Management and Research 2009, 27:603-610.

1. A method for preparing an insect biomass, comprising:
i) forming a composition comprising at least one lignocellulose digesting fungal cell and/or spore and at least one lignocellulose substrate;
ii) incubating the composition of i) to obtain a digested or partially digested lignocellulose substrate to form a pre-treated lignocellulose substrate; and
iii) contacting the pre-treated lignocellulose substrate with at least one insect species egg or larvae to provide insect biomass; and optionally iv) harvesting said insect biomass.

2. The method according to claim 1 wherein said at least one lignocellulose substrate is particulate.

3. The method according to claim 1 wherein said method further comprises lyophilisation of the pre-treated lignocellulose substrate or freezing the pre-treated lignocellulose substrate.

4. (canceled)

5. The method according to claim 1, wherein said pre-treated lignocellulose substrate is adjusted to a moisture content of at least 40 w/v %.

6. The method according to claim 1, wherein said insect biomass
is separated from said pre-treated lignocellulose substrate and milled to form a feed, or
is combined with pre-treated lignocellulose substrate and milled to form a composite feed.

7. (canceled)

8. The method according to claim 1, wherein said fungal cell and/or spore is an Ascomycete fungal species.

9. The method according to claim 8 wherein said Ascomycete species is Parascedosporium sp.

10. The method according to claim 9 wherein said Parascedosporium sp. is Parascedosporium putredinis.

11. The method according to claim 10 wherein said Parascedosporium putredinis is Parascedosporium putredinis NO1 deposited under accession number IMI 507228.

12. The method according to claim 1, wherein said at least one lignocellulose substrate comprises lignocellulose obtained from wheat straw, rice straw, corn stover, wheat bran, oat bran, oilseed rape straw, miscanthus straw, willow, sugarcane bagasse, oil palm empty fruit bunch, cacao husks, or any combination thereof.

13. (canceled)

14. The method according to claim 1, wherein said at least one lignocellulose substrate is supplemented with fungal growth medium or a nitrogen source.

15.-16. (canceled)

17. The method according to claim 1, wherein said insect biomass is derived from house fly egg or larvae (Musca domestica), black soldier fly egg or larvae (Hermetia illucens), mealworms (Tenebrio molitor), locust egg or nymph (Locusta migratoria, Schistocerca gregaria, Oxya sp.), silkworms (Bombyx mori), crickets (Acheta domesticus and Teleogryllus commodus), or cockroach (Periplaneta americana).

18.-19. (canceled)

20. An insect feed or animal feed comprising the insect biomass obtained by the method according to claim 1.

21. (canceled)

22. A method for preparing an insect biomass or animal feed, comprising using a lignocellulose digesting fungal cell to obtain a digested or partially digested lignocellulose substrate, wherein said fungal cell comprises:
i) a nucleic acid sequence that hybridises under high stringency conditions with a nucleic acid probe complementary to the nucleotide sequence set forth in SEQ ID NO: 1;
ii) a nucleotide sequence comprising 95%, 96%, 97%, 98%, 99%, or 100% nucleotide sequence identity to the full-length sequence set forth in SEQ ID NO: 1; and/or
iii) a nucleic acid sequence that can be amplified by a polymerase chain reaction using oligonucleotide primer pairs comprising or consisting of

| (SEQ ID NO: 2) | |
|---|---|
| | TCCGTAGGTGAACCTGCGG |
| | and |
| (SEQ ID NO: 3) | |
| | CGCTGCGTTCTTCATCG. |

23.-26. (canceled)

27. The method according to claim 22 wherein said fungal cell is an Ascomycete fungal species.

28.-30. (canceled)

31. A lignocellulose digesting fungal strain Parascedosporium putredinis NO1 deposited under accession number IMI 507228.

32. A bioreactor comprising: at least one lignocellulose digesting fungal cell and/or spore and at least one lignocellulose substrate and at least one insect species egg or larvae to provide insect biomass.

33. The bioreactor according to claim 32,
wherein said lignocellulose digesting fungal cell comprises:
i) a nucleic acid sequence that hybridises under high stringency conditions with a nucleic acid probe complementary to the nucleotide sequence set forth in SEQ ID NO: 1; or
ii) a nucleotide sequence comprising 95%, 96%, 97%, 98% or 99% nucleotide sequence identity to the full-length sequence set forth in SEQ ID NO: 1; and/or
wherein said fungal said lignocellulose digesting fungal cell is characterised by at least one amplicon amplified by a polymerase chain reaction wherein said reaction comprises oligonucleotide primer pairs comprising or consisting of:

| (SEQ ID NO: 2) | |
|---|---|
| | TCCGTAGGTGAACCTGCGG |
| | and |
| (SEQ ID NO: 3) | |
| | CGCTGCGTTCTTCATCG. |

34. (canceled)

35. The bioreactor according to claim 32 wherein said lignocellulose digesting fungal cell is Parascedosporium putredinis.

36. (canceled)

37. A cell culture comprising cells of the fungal strain of claim 31, wherein said culture is substantially a monoculture of said fungal strain.
